(19) United States
(12) Patent Application Publication
(10) Pub. No.: US 2010/0256402 A1
(43) Pub. Date: Oct. 7, 2010

(54) NOVEL SOLVATE

(75) Inventors: Andreas Hotter, Woergl (AT); Josef Wieser, Polling (AT); Arthur Pichler, Buch bei Jenbach (AT)

Correspondence Address: Manelii Denison & Selter PLLC, 2000 M Street, 7th Floor, Washington DC, DC, 20036, US

(73) Assignee: Sandoz AG, Basel (CH)

(21) Appl. No.: 12/742,690

(22) PCT Filed: Nov. 12, 2008
(86) PCT No.: PCT/EP2008/065401

§ 371 Date: May 13, 2010

(30) Foreign Application Priority Data
Nov. 14, 2007 (EP) 07120674.2

(51) Int. Cl. C07C 237/26

(52) U.S. Cl. 552/205

(57) ABSTRACT

The present invention relates to the novel crystalline form X of Tigecycline and to processes of preparing the same. Furthermore the present invention relates to the use of crystalline form X of Tigecycline as an intermediate for the preparation of an anti-infective medicament. Moreover the present invention relates to the use of crystalline form X of Tigecycline for the preparation of acid addition salts of Tigecycline

### FIELD OF THE INVENTION

The present invention relates to the novel crystalline form X of Tigecycline and to processes of preparing the same. Furthermore the present invention relates to the use of crystalline form X of Tigecycline as an intermediate for the preparation of an anti-infective medicament. Moreover the present invention relates to the use of crystalline form X of Tigecycline for the preparation of acid addition salts of Tigecycline.

### BACKGROUND OF THE INVENTION

Tigecycline, (4S,4aS,5aR,12aS)-4,7-Bis(dimethylamino)-9-[[[(1,1-dimethylethyl)amino]acetyl]amino]-1,4,4a,5,5a,6,11,12a-octahydro-3,10,12,12a-tetrahydroxy-1,11-dioxo-2-naphthacenecarboxamide, is a 9-t-butylglycylamido derivative of minocycline (Merck Index 14^{th} Edition, monograph number 9432, CAS Registry Number 220620-09-7). Compared to other tetracycline antibiotics Tigecycline is more active against tetracycline-resistant strains and also more tolerable. Tigecycline possesses activity against bacterial isolates containing the two major determinants responsible for tetracycline-resistance: ribosomal protection and active efflux of the drug out of the bacterial cell. Furthermore Tigecycline possesses broad spectrum activity, e.g. it is active against gram-positive pathogens (e.g. methicillin-resistant Staphylococcus aureus, vancomycin-resistant Enterococci), gram-negative pathogens (e.g. Acinetobacter baumannii, Stenotrophomonas maltophilia) and anaerobic pathogens. At the moment Tigecycline is indicated for the treatment of complicated skin and soft-tissue infections and intra-abdominal infections. (P. J. Petersen et al., Antimicrob. Agents Chemoth. 1999; 43: 738-744. R. Patel et al., Diagnostic Microbiology and Infectious Disease 2000; 38: 177-179. H. W. Boucher et al., Antimicrob. Agents Chemoth. 44: 2225-2229. D. J. Biedenbach et al., Diagnostic Microbiology and Infectious Disease 2001; 40: 173-177. P. J. Petersen et al., Antimicrob. Agents Chemoth. 2002; 46: 2595-2601. D. Milatovic et al., Antimicrob. Agents Chemoth. 47: 400-404. T. Hirata et al., Antimicrob. Agents Chemoth. 2004; 48: 2179-2184. G. A. Pankey, Journal of Antimicrobial Chemotherapy 2005; 56: 470-480. R. Harris et al., P&T 2006; 31: 18-59.). Tigecycline is available on the market as lyophilized powder for injection, the originator is Wyeth. During the formulation process Tigecycline is first dissolved in water and then lyophilized. Therefore a crystalline form of Tigecycline should show appropriate water solubility.

When a crystalline form of Tigecycline is used for finished dosage form production, the form should show suitable stability and hygroscopicity properties as well. Tigecycline form X fulfills all these requirements and is therefore a suitable form for preparing an anti-infective medicament.

Patent application WO 2006/128150 discloses crystalline forms I to V of Tigecycline and methods of their preparation. Patent application WO 2007/127292 discloses two additional crystalline forms (I and II) of Tigecycline and methods of their preparations. Nevertheless, there remains a need for alternative crystalline forms of Tigecycline with suitable water solubility, purity, stability and hygroscopicity properties. In addition the crystalline forms should be straight forward to prepare and crystallize in essentially pure crystalline form.

### SUMMARY OF THE INVENTION

In one embodiment the present invention refers to crystalline form X of Tigecycline. Crystalline form X of Tigecycline can be described by an X-ray powder diffraction pattern comprising peaks at 2-theta angles of 4.9°±0.2°, 9.0°±0.2°, 10.0°±0.2°, 12.7°±0.2°, 13.6°±0.2°, 15.1°±0.2°, 16.1°±0.2°, 16.9°±0.2°, 18.4°±0.2°, 19.1°±0.2°, 20.2°±0.2°, 21.6°±0.2° and 23.8°±0.2°.

Alternatively crystalline form X of Tigecycline can be described by an infrared spectrum comprising peaks at wavenumbers of 3376±2 cm⁻¹, 2961±2 cm⁻¹, 1674±2 cm⁻¹, 1588±2 cm⁻¹, 1530±2 cm⁻¹, 1415±2 cm⁻¹, 1365±2 cm⁻¹, 1284±2 cm⁻¹, 1212±2 cm⁻¹, 1181±2 cm⁻¹, 1102±2 cm⁻¹, 1053±2 cm⁻¹, 1022±2 cm⁻¹, 994±2 cm⁻¹, 973±2 cm⁻¹, 872±2 cm⁻¹, 803±2 cm⁻¹, 693±2 cm⁻¹ and 653±2 cm⁻¹.

A first process for the preparation of crystalline form X of Tigecycline comprising the steps of:
a) slurrying Tigecycline in 2-butanol at room temperature;
b) stirring the slurry at room temperature or below to effect transformation of the suspended form into form X; and
c) isolating crystalline form X of Tigecycline;
   is also subject matter of the present invention.

In addition the present invention provides a second process for the preparation of form X of Tigecycline comprising the steps of:
a) dissolving Tigecycline in 2-butanol at 30° C. to 99° C.;
b) slowly cooling down the solution to room temperature or below to effect crystallization; and
c) isolating crystalline form X of Tigecycline;

Furthermore the present invention relates to the use of crystalline form X of Tigecycline as an intermediate for preparing an anti-infective medicament.

Moreover the present invention relates to the use of form X of Tigecycline for the preparation of acid addition salts of Tigecycline.

Other objects, features, advantages and aspects of the present invention will become apparent to those of skill from the following description. It should be understood, however, that the description and the following specific examples, while indicating preferred embodiments of the invention, are given by way of illustration only. Various changes and modifications within the spirit and scope of the disclosed invention will become readily apparent to those skilled in the art from reading the description and from reading the other parts of the present disclosure.

### BRIEF DESCRIPTION OF THE DRAWINGS

FIG. 1: X-ray powder diffraction pattern of form X of Tigecycline

FIG. 2: Infrared spectrum of form X of Tigecycline

FIG. 3: Differential scanning calorimetric curve of form X of Tigecycline

FIG. 4: Thermogravimetric analysis curve of form X of Tigecycline

### DETAILED DESCRIPTION OF THE INVENTION

The inventors of the present invention have identified a novel crystalline form of Tigecycline. The chemical structure of Tigecycline is displayed in Figure A. The novel crystalline form is a solvate, and may be characterized e.g. by a typical X-ray powder diffraction pattern, an infrared spectrum, a characteristic differential scanning calorimetric (DSC) curve or by thermogravimetric analysis (TGA). Each of these characteristics on its own is sufficient to unambiguously define and identify the new crystalline form but they also may be combined with each other.

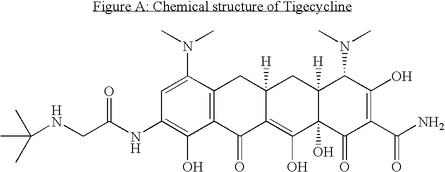

The present invention relates to the novel crystalline form X of Tigecycline. Form X of Tigecycline is a 2-butanol solvate, hereinafter also referred to as “form X” characterized by an X-ray powder diffraction pattern with peaks at 2-theta angles of 4.9°±0.2°, 9.0°±0.2°, 10.0°±0.2°, 12.7°±0.2°, 13.6°±0.2°, 15.1°±0.2°, 16.1°±0.2°, 16.9°±0.2°, 18.4°±0.2°, 19.1°±0.2°, 20.2°±0.2°, 21.6°±0.2° and 23.8°±0.2°.

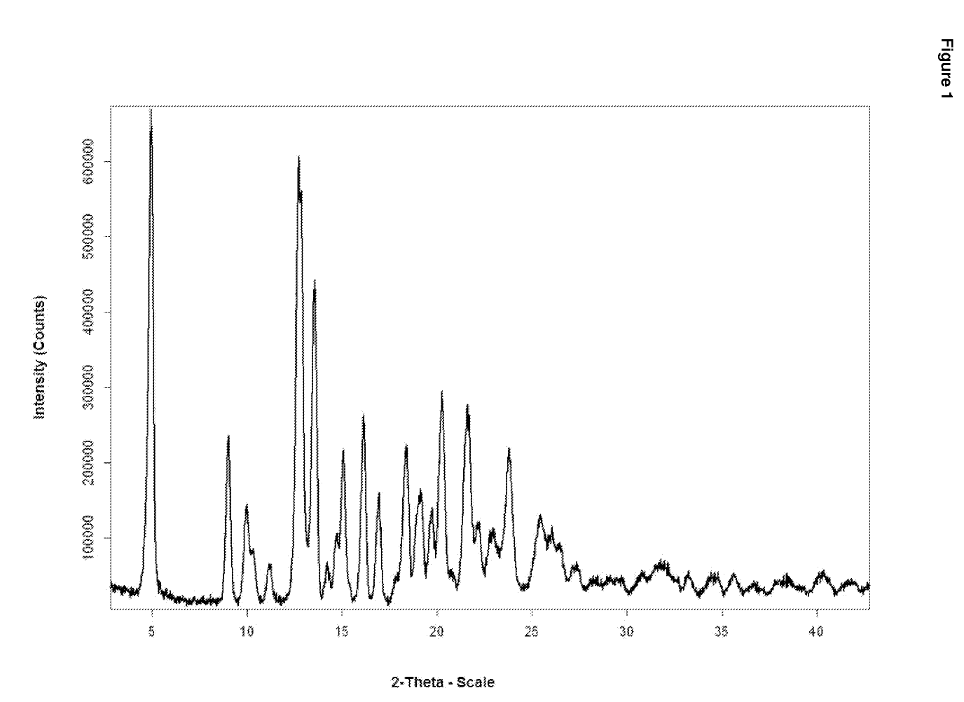

A characteristic X-ray powder diffraction pattern of form X of Tigecycline is shown in FIG. 1 and some characteristic peaks are listed in Table 1.

Accordingly, in a preferred embodiment, the present invention relates to a novel form X of Tigecycline characterized by an X-ray powder diffraction pattern substantially in accordance with Table 1 and FIG. 1.

The X-ray powder diffraction pattern of form X of Tigecycline clearly can be distinguished from these of form Ito V disclosed in patent application WO 2006/150128 and from these of forms I and II disclosed in patent application WO 2007/127292. Thus form X of Tigecycline can be seen as a novel crystalline form of Tigecycline.

TABLE 1
Characteristic X-Ray Powder Diffraction
(XRPD) peaks of form X of Tigecycline

| Angle | relative Intensity |
|---|---|
| [°2-theta] | [%] |
| 4.9 | 100.0 |
| 9.0 | 35.3 |
| 10.0 | 21.7 |
| 12.7 | 90.7 |
| 13.6 | 66.2 |
| 15.1 | 32.5 |
| 16.1 | 39.5 |
| 16.9 | 24.1 |
| 18.4 | 33.5 |
| 19.1 | 24.8 |
| 20.2 | 44.1 |
| 21.6 | 41.5 |
| 23.8 | 32.8 |

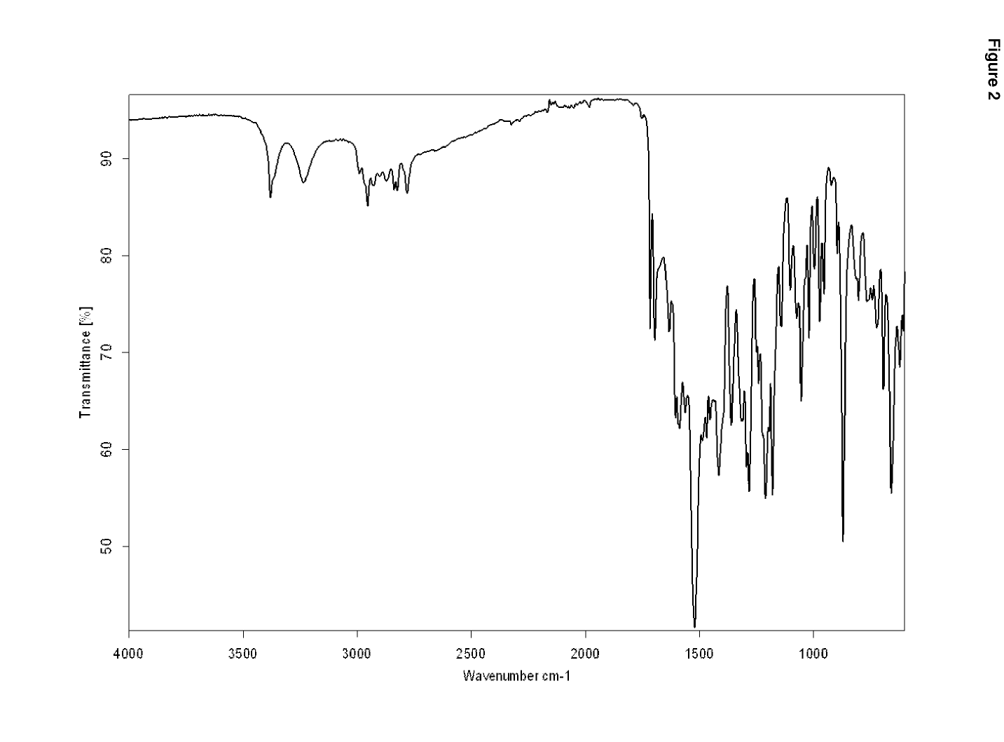

Form X of Tigecycline also may be characterized by a typical infrared spectrum as shown in FIG. 2. Accordingly, in a further preferred embodiment, the present invention relates to form X of Tigecycline characterized by an infrared spectrum substantially in accordance with FIG. 2. Characteristic bands are present at wavenumbers of 3376±2 cm⁻¹, 2961±2 cm⁻¹, 1674±2 cm⁻¹, 1588±2 cm⁻¹, 1530±2 cm⁻¹, 1415±2 cm⁻¹, 1365±2 cm⁻¹, 1284±2 cm⁻¹, 1212±2 cm⁻¹, 1181±2 cm⁻¹, 1102±2 cm⁻¹, 1053±2 cm⁻¹, 1022±2 cm⁻¹, 994±2 cm⁻¹, 973±2 cm⁻¹, 872±2 cm⁻¹, 803±2 cm⁻¹, 693±2 cm⁻¹ and 653±2 cm⁻¹.

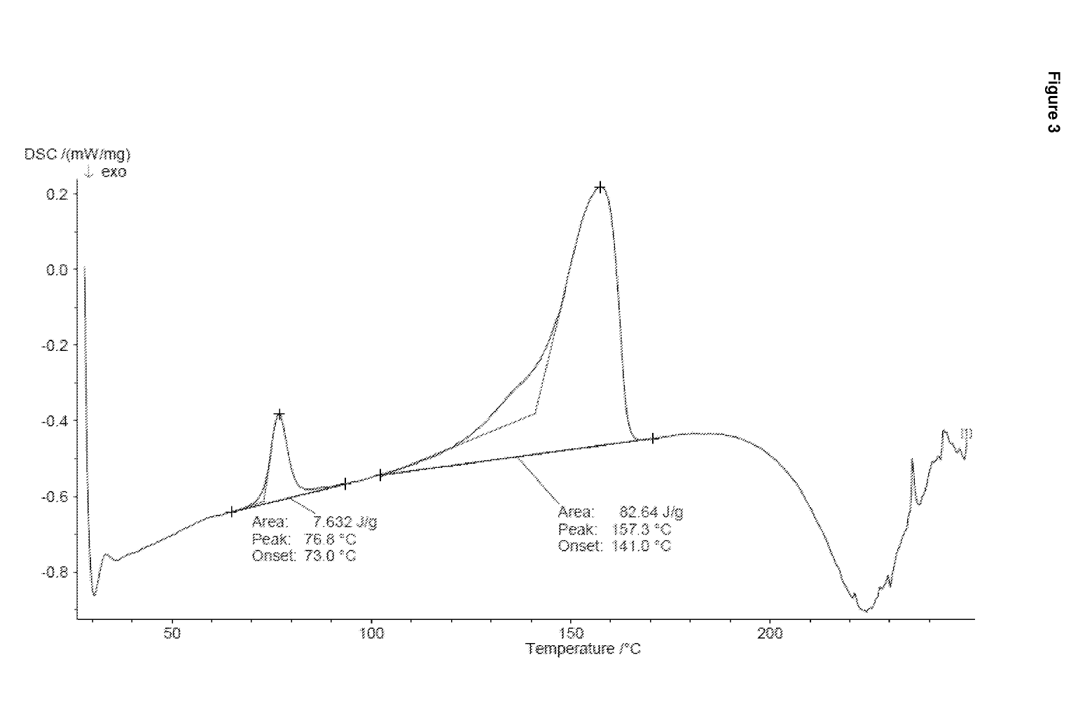

In addition, form X of Tigecycline shows a characteristic DSC curve at a heating rate of 10° C./min. The DSC curve in FIG. 3 displays two endothermic peaks with maxima at about 77° C. respectively 157° C.

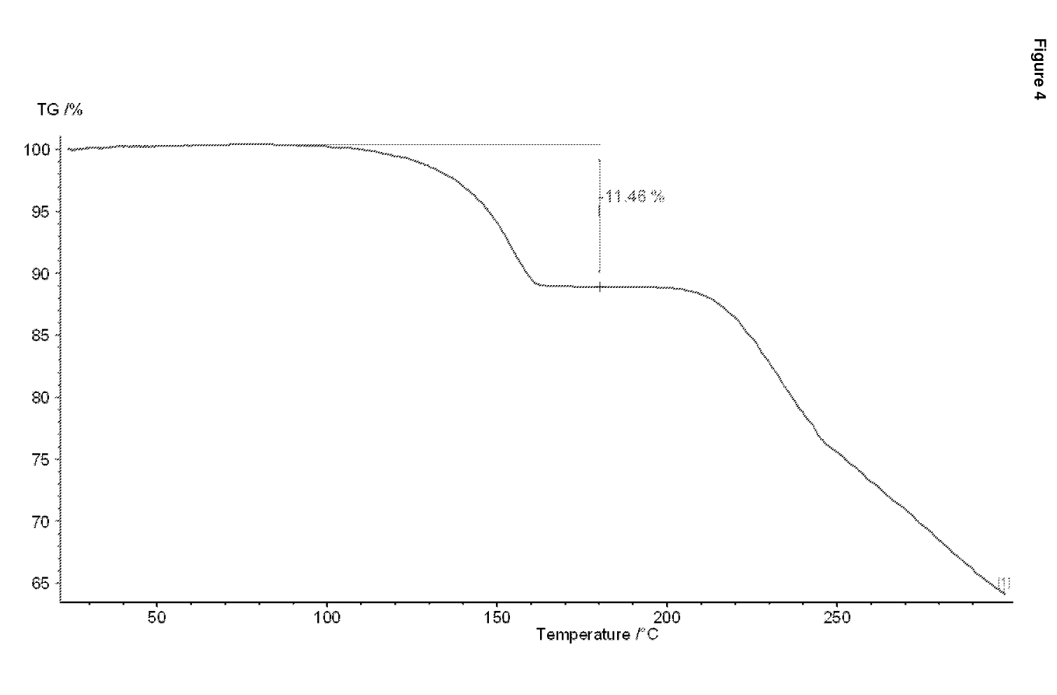

FIG. 4 shows the TGA curve of form X of Tigecycline, which displays a total weight loss of about 11.5%, due to the desolvation process.

Form X of Tigecycline is a 2-butanol monosolvate as a batch of Tigecycline form X showed a 2-butanol content of 1.09 mol by GC and 1.02 mol by TGA.

In one embodiment, the present invention provides a first process for the preparation of form X of Tigecycline, comprising the steps of:
a) slurrying Tigecycline in 2-butanol at room temperature;
b) stirring the slurry at room temperature or below to effect transformation of the suspended form of Tigecycline into form X; and
c) isolating crystalline form X of Tigecycline;

In step a) Tigecycline is preferably slurried at a concentration ranging from 5 to 400 g/L, more preferably ranging from 5 to 200 g/L, most preferably ranging from 5 to 100 g/L.

In step b) it is crucial that the temperature is chosen such that the used form of Tigecycline remains in the condition of a suspension and does not become dissolved. That's why room temperature or even lower temperature is applied. Optionally, the method can further comprise seeding the slurry with Tigecycline form X.

Furthermore the present invention provides a second process of the preparation of form X of Tigecycline, comprising the steps of:
a) dissolving Tigecycline in 2-butanol at 30 to 99° C.;
b) slowly cooling down the solution to room temperature or below to effect crystallization; and
c) isolating crystalline form X of Tigecycline;

The concentration of Tigecycline in step a) preferably ranges from 5 to 100 g/L, more preferably from 5 to 40 g/L, most preferably from 5 to 20 g/L.

The temperature in step b) may be in the range from 30 to 99° C., depending on the form and concentration of Tigecycline used. However the temperature should be chosen such that a clear solution is obtained.

“Slow cooling” as mentioned in step c) means in this special case a decrease in temperature from e.g. the boiling point of 2-butanol to 0 to 5° C. preferably within 1 to 24 hours, more preferably within 2 to 12 hours, most preferably within 3 to 6 hours.

The crystallization step c) of the above process may be facilitated by adding seed crystals of form X of Tigecycline.

For preparing form X of Tigecycline according to the above processes, any form of Tigecycline may be used, e.g. the amorphous form, crystalline form I to V disclosed in WO 2006/128150 or crystalline forms I and II of WO 2007/127292. In addition, also forms of low crystallinity or mixtures of two or more different forms of Tigecycline are suitable.

The processes represent practical methods of purifying Tigecycline, because most of the impurities of Tigecycline are more soluble in 2-butanol and remain in solution. For example Tigecycline (1.9% total impurities, with a C₄-epimer content of 1.4%) was recrystallized with 2-butanol to obtain the 2-butanol solvate in high purity (0.6% total impurities, with a C₄-epimer content of 0.2%).

In addition form X of Tigecycline is also a particularly suitable form for the isolation of Tigecycline in the last step of the synthesis of Tigecycline. If, for example 9-chloroacetaminominocycline is reacted with tert.-butylamine in dimethylacetamide Tigecycline can be obtained after a simple extractive work up in high yield and in high purity without an additional purification step.

The inventors of the present invention found a novel crystalline form of Tigecycline, namely form X, with suitable properties for the preparation of an anti-infective medicament.

After storing the different crystalline forms of Tigecycline for 7 days at 80° C., form X of the present invention clearly shows higher stability than e.g. form I and form II of WO 2006/128150 which is displayed in Table 2. Form I and form II of WO 2006/128150 show a tremendous increase in both, total impurities and 4-Epi-Tigecycline content, and are consequently of disadvantage when these forms are used as intermediates in finished dosage form production.

Furthermore suitable crystalline forms of Tigecycline should be low hygroscopic, as water uptake may cause the formation of undesired byproducts like e.g. 4-Epi-Tigecycline. Table 3 displays the water uptake of the different crystalline forms of Tigecycline after open storage for 24 hours at 80% relative humidity. For example form III of WO 2006/128150 shows a water uptake of 7.32%, form II of WO 2006/128150 of 7.11% and form I of WO 2007/127292 of 6.26%. Hence these forms are not suitable for the use as intermediates in finished dosage form production. On the contrary crystalline form X of the present invention shows a water uptake of 0.57%. Therefore crystalline form X is less hygroscopic than forms I, II, Ill and V of WO 2006/128150 and also less hygroscopic than form I of WO 2007/127292.

During the formulation process Tigecycline undergoes a lyophilization process, where the active substance is dissolved in water before lyophilizing. Hence crystalline forms of Tigecycline are required to show suitable water solubility. As displayed in Table 4 form I and form II of WO 2007/127292 clearly show the worst water solubility of all forms and are therefore not the first choice for the lyophilizing step. Although form X of the present invention does not show the highest solubility, the value is appropriate for lyophilizing. For example form X shows higher water solubility than form V of WO 2006/128150.

Moreover crystalline form X of Tigecycline is straight forward to prepare and obtained in pure crystalline form by the processes described above. In contrast form IV of WO 2006/128150 couldn't be crystallized by the inventors of the present invention when repeating examples 7 and 8 of WO 2006/128150, which describe processes for the preparation of form IV. In our hands form IV of WO 2006/128150 is not reproducible by the methods described.

TABLE 2
Stability data after storing different forms of Tigecycline at 80° C. for 7 days

| | 4-Epi- | | | |
|---|---|---|---|---|
| | Tigecycline | 4-Epi- | Total impurities | |
| | ambient | Tigecycline | ambient | Total impurities |
| Form | conditions | 7 days at 80° C. | conditions | 7 days at 80° C. |
| WO 2006/128150 | | | | |
| I | 0.36 | 2.15 | 0.75 | 11.20 |
| II | 1.04 | 10.83 | 1.60 | 18.44 |
| III | 0.16 | 2.18 | 0.16 | 4.46 |
| IV | 0.23 | 0.38 | 0.40 | 0.87 |
| V | <0.05 | 0.18 | 0.22 | 0.37 |
| WO 2007/127792 | | | | |
| I | 0.34 | 1.26 | 0.59 | 2.22 |
| II | 0.17 | 0.38 | 0.24 | 0.91 |
| Present invention | | | | |
| X | 0.20 | 0.79 | 0.58 | 3.48 |

TABLE 3
Water content and hygroscopicity data
of different forms of Tigecycline

| | water | | |
|---|---|---|---|
| | content [%] | | |
| | ambient | water content | water uptake |
| Form | conditions | 1 day at 80% rH | 1 day at 80% rH |
| WO 2006/128150 | | | |
| I | 2.01 | 5.01 | 3.00 |
| II | 2.93 | 10.04 | 7.11 |
| III | 0.69 | 8.01 | 7.32 |
| IV | 2.90 | 3.33 | 0.43 |
| V | 0.19 | 1.88 | 1.69 |
| WO 2007/127292 | | | |
| I | 0.13 | 6.39 | 6.26 |
| II | 0.18 | 0.25 | 0.07 |
| Present invention | | | |
| X | 0.29 | 0.86 | 0.57 |

TABLE 4
Solubility data of different forms of Tigecycline

| | | Base used |
|---|---|---|
| Form | Concentration [mg/ml] | [mg base equivalent] |
| WO 2006/128150 | | |
| I | 201 | 206 |
| II | 170 | 280 |
| III | 174 | 198 |
| IV | 322 | 387 |
| V | 134 | 180 |
| WO 2007/127292 | | |
| I | 54 | 156 |
| II | 76 | 198 |
| Present invention | | |
| X | 162 | 172 |

To sum it up crystalline form X of Tigecycline is a particularly suitable form as an intermediate for the formulation of an anti-infective medicament due to appropriate properties like physical stability, water solubility, hygroscopicity and purity. Furthermore the processes for preparing form X of the present invention are reproducible and suitable for pharmaceutical scale-up.

In addition crystalline form X of Tigecycline is a particularly suitable form for the preparation of acid addition salts, e.g. pharmaceutical acceptable addition salts of Tigecycline e.g. mono- and dihydrochlorides, solvates and hydrates thereof, useful as medicaments.

The invention is further described by reference to the following examples. These examples are provided for illustration purposes only and are not intended to be limiting the present invention in any way.

### EXAMPLES

The X-ray powder diffraction pattern (XRPD) was collected on a Unisantis XMD 300 X-ray powder diffractometer with a position sensitive detector in parallel beam optics using the following acquisition conditions: tube anode: Cu, 40 kV, 0.8 mA; 3-43° theta/2theta; simultaneous detection of regions of 10° per step with detector resolution 1024, counting time 300 seconds per step. The sample was measured at room temperature in a standard sample holder on a rotating sample spinner. A typical precision of the 2-theta values is in the range of ±about 0.2° 2-Theta. Thus a diffraction peak that appears at 5.0° 2-Theta can appear between 4.8 and 5.2° 2-Theta on most X-ray diffractometers under standard conditions.

The infrared spectrum (IR) was collected on a MKII Golden Gate™ Single Reflection Diamond ATR (attenuated total reflection) cell with a Bruker Tensor 27 FTIR spectrometer with 4 cm⁻¹ resolution at ambient conditions. To collect a spectrum a spatula tip of a sample was applied to the surface of the diamond in powder form. Then the sample was pressed onto the diamond with a sapphire anvil and the spectrum was recorded. A spectrum of the clean diamond was used as background spectrum. A typical precision of the wavenumber values is in the range of about ±2 cm⁻¹. Thus, an infrared peak that appears at 1716 cm⁻¹ can appear between 1714 and 1718 cm⁻¹ on most infrared spectrometers under standard conditions.

Differential scanning calorimetry (DSC) was performed on a Netzsch DSC 204. About 4 mg sample was heated in 25 μl Al-Pans with loose lids from room temperature to 250° C. at a rate of 10° C./min. Nitrogen (purge rate 20 ml/min) was used as purge gas.

Thermogravimetric analysis (TGA) was performed on a Netzsch STA 409 PC/PG instrument. Samples were heated in an Al2O3 crucible from room temperature to 300° C. at a rate of 10° C./min. Nitrogen (purge rate 50 ml/min) was used as purge gas.

### Example 1

### Preparation of Form X

A suspension of 570 mg Tigecycline in 6 ml 2-butanol was stirred at room temperature for about 2 h. The solid was filtered off, washed with 2-butanol and dried under vacuum at room temperature to obtain 571 mg (100% yield) of crystalline form X of Tigecycline (99.47% purity by HPLC).

### Example 2

### Preparation of Form X

A suspension of 5.00 g Tigecycline in 50 ml 2-butanol was stirred at room temperature. After the addition of seed crystals of form X obtained from example 1 the suspension was stirred for 4 hours. The solid was filtered off, washed with 2-butanol and dried under vacuum at room temperature to obtain 4.80 g (101% yield) of crystalline form X of Tigecycline (99.23% purity by HPLC).

### Example 3

### Preparation of Form X

500 mg Tigecycline were slurried in 25 ml 2-butanol at room temperature. The suspension was heated (80° C. bath temperature) to obtain a clear solution. The solution was allowed to cool down slowly (within 150 minutes) to room temperature while gently stirring. Then the oil bath was replaced by an ice/water bath to effect complete crystallization. Finally the solid was filtered off, washed with acetone and dried under vacuum at room temperature to obtain 379 mg of crystalline form X of Tigecycline.

### Example 4

### Preparation of Form X

A solution of 50.0 mg Tigecycline (Tygacil® 50 mg, Wyeth®, powder for infusion) in 500 μl 2-butanol is stirred at room temperature. After about 3 minutes an orange precipitate is obtained and the suspension is stirred for 1 hour at room temperature. The solid is filtered off, washed with 2-butanol and dried under vacuum at room temperature for 17 hours to obtain 54.4 mg of the crystalline form X.

### Example 5

### Water Solubility Testing

A UV-vis Lambda 35 spectrophotometer (Perkin-Elmer) was used (A=347 nm, 1.0 cm quartz cells). Perkin Elmer® UV WinLab-5.1 software was used.

A saturated solution of Tigecycline in distilled water was prepared and the suspension was stirred at room temperature for 30 minutes with a stirring speed of 1000 U/min. The suspension was filtrated through a 0.45 μm filter. Finally the resulting solution was diluted 10000-fold and measured against water at a wavelength of 347 nm. The results are listed in the following table:

| | | Base used |
|---|---|---|
| Form | Concentration [mg/ml] | [mg base equivalent] |
| WO 2006/128150 | | |
| I | 201 | 206 |
| II | 170 | 280 |
| III | 174 | 198 |
| IV | 322 | 387 |
| V | 134 | 180 |
| WO 2007/127292 | | |
| I | 54 | 156 |
| II | 76 | 198 |
| Present invention | | |
| X | 162 | 172 |

1-11. (canceled)

12. A crystalline form X of Tigecycline.

13. The crystalline form X of Tigecycline according to claim 12, having an X-ray powder diffraction pattern comprising peaks at 2-theta angles of 4.9°±0.2°, 9.0°±0.2°, 10.0°±0.2°, 12.7°±0.2°, 13.6°±0.2°, 15.1°±0.2°, 16.1°±0.2°, 16.9°±0.2°, 18.4°±0.2°, 19.1°±0.2°, 20.2°±0.2°, 21.6°±0.2° and 23.8°±0.2°.

14. The crystalline form X of Tigecycline according to claim 12, having an X-ray powder diffraction pattern with a peak at position 4.9°±0.2° 20 having the highest relative intensity.

15. The crystalline form X of Tigecycline according to claim 12, having an infrared spectrum comprising peaks at wavenumbers of 3376±2 cm⁻¹, 2961±2 cm⁻¹, 1674±2 cm⁻¹, 1588±2 cm⁻¹, 1530±2 cm⁻¹, 1415±2 cm⁻¹, 1365±2 cm⁻¹, 1284±2 cm⁻¹, 1212±2 cm⁻¹, 1181±2 cm⁻¹, 1102±2 cm⁻¹, 1053±2 cm⁻¹, 1022±2 cm⁻¹, 994±2 cm⁻¹, 973±2 cm⁻¹, 872±2 cm⁻¹, 803±2 cm⁻¹, 693±2 cm⁻¹ and 653±2 cm⁻¹.

16. The crystalline form X of Tigecycline according to claim 12, having an X-ray powder diffraction pattern substantially in accordance FIG. 1.

17. The crystalline form X of Tigecycline according to claim 12, having an infrared spectrum substantially in accordance with FIG. 2.

18. The crystalline form X of Tigecycline according to claim 12, having a differential scanning calorimetric curve having two endothermic peaks with maxima at about 77° C. and 157° C., when heated at a rate of 10° C. per minute.

19. The crystalline form X of Tigecycline according to claim 12, having a differential scanning calorimetric curve substantially in accordance with FIG. 3.

20. The crystalline Form X of Tigecycline according to claim 12, having a thermogravimetric analysis curve substantially in accordance with FIG. 4.

21. The crystalline Form X of Tigecycline according to claim 12, wherein the crystalline form comprises a 2-butanol monosolvate.

22. The crystalline Form X of Tigecycline according to claim 12, having enhanced storage stability compared to forms I and II of Tigecycline.

23. The crystalline Form X of Tigecycline according to claim 12, having a lower hygroscopic compared to forms I, II, III, and V of Tigecycline.

24. A process of preparing crystalline form X of Tigecycline comprising the steps of:
a) slurrying Tigecycline in 2-butanol at room temperature to form a suspension;
b) stirring the suspension at room temperature or below to effect transformation of the Tigecycline into form X; and
c) isolating crystalline form X of Tigecycline.

25. The process of preparing crystalline Form X of Tigecycline according to claim 24, wherein in step a) the Tigecycline is slurried at a concentration of from 5 to 400 g/L.

26. The process of preparing crystalline Form X of Tigecycline according to claim 24, wherein in step a) the Tigecycline is slurried at a concentration of from 5 to 100 g/L.

27. The process of preparing crystalline Form X of Tigecycline according to claim 24, wherein in step b) the temperature is such that the Tigecycline remains in suspension and does not become dissolved in the 2-butanol.

28. A process of preparing crystalline form X of Tigecycline comprising the steps of:
a) dissolving Tigecycline in 2-butanol at 30 to 99° C. to form a solution;
b) slowly cooling down the solution to room temperature or below to effect crystallization; and
c) isolating crystalline form X of Tigecycline.

29. The process of preparing crystalline Form X of Tigecycline according to claim 28, wherein in step a) the concentration of Tigecycline is from 5 to 100 g/L.

30. The process of preparing crystalline Form X of Tigecycline according to claim 28, wherein in step a) the concentration of Tigecycline is from 5 to 20 g/L.

31. The process of preparing crystalline Form X of Tigecycline according to claim 28, wherein in step b) the temperature is such that the solution is clear.

32. The process of preparing crystalline Form X of Tigecycline according to claim 28, wherein the slow cooling in step b) comprises decreasing the temperature of the solution to 0 to 5° C. over a period within 1 to 24 hours.

33. The process of preparing crystalline Form X of Tigecycline according to claim 28, wherein the slow cooling in step b) comprises decreasing the temperature of the solution to 0 to 5° C. over a period within 2 to 12 hours.

34. The process of preparing crystalline Form X of Tigecycline according to claim 28, wherein the slow cooling in step b) comprises decreasing the temperature of the solution to 0 to 5° C. over a period within 3 to 6 hours.

35. A method comprising using crystalline form X of Tigecycline in the manufacture of a sterile lyophilized composition for use as a medicament.

36. A method comprising using crystalline form X of Tigecycline for the preparation of an anti-infective medicament.

37. A method of using crystalline form X of Tigecycline in the purification of Tigecycline.

38. A method of using crystalline form X of Tigecycline in the preparation of acid addition salts.
